(19) United States
(12) Patent Application Publication
(10) Pub. No.: US 2025/0256097 A1
(43) Pub. Date: Aug. 14, 2025

(54) METHODS AND DEVICE FOR PROMOTING BONE GROWTH BY ELECTRICAL STIMULATION

(71) Applicant: KAT INNOVATION INC., Montreal (CA)

(72) Inventors: Jean P. Boucher, Beloeil (CA); Alain Steve Comtois, St-Lazare (CA); James A. Hodgdon, San Diego, CA (US)

(73) Assignee: KAT INNOVATION INC., Montreal, QC (CA)

(21) Appl. No.: 18/548,063

(22) PCT Filed: Feb. 25, 2022
(86) PCT No.: PCT/IB2022/051697

§ 371 Date: Aug. 25, 2023

Related U.S. Application Data
(60) Provisional application No. 63/154,245, filed on Feb. 26, 2021.

(51) Int. Cl. A61N 1/36; A61N 1/04; A61N 1/32

(52) U.S. Cl. CPC A61N 1/36034; A61N 1/0464; A61N 1/0484; A61N 1/0496; A61N 1/326

(57) ABSTRACT

A method for preventing bone loss. promoting bone growth, or healing a bone in a subject includes directly electrically stimulating bone cells with a low-intensity electrical current avoiding muscle contraction, administering the electrical stimulation with the low-intensity electrical current in a manner which prevents bone loss or heals bones. The bone cells are selected from a group consisting of arms, legs, pelvis, ribs, trunk. head, spine, wrist, ankle and whole body. The electrical stimulation with the low-intensity electrical current provides at least one of the following benefits: maintenance of bone quality or bone health prevention of osteoporosis, treatment of osteoporosis, promotion of bone healing, prevention of fractures, treatment of fractures, treatment of spinal fusion, and treatment of spinal-cord iniury.

### CROSS REFERENCE TO RELATED APPLICATIONS

The present application claims the priority benefits of International Patent Application No. PCT/IB2022/051697, filed on Feb. 25, 2022, and claims benefit of U.S. Provisional Application 63/154,245, filed on filed on Feb. 26, 2021, which are hereby incorporated herein by reference in their entireties.

### FIELD OF THE INVENTION

The invention relates to the field of electro-medicine, and more particularly to methods and devices for promoting bone growth by using electrical stimulations.

### BACKGROUND OF THE INVENTION

More than 200 million women are suffering from osteoporosis worldwide, the disease affecting I woman out of 3. The yearly medical costs associated to osteoporosis are about 17 billion dollars in USA and 4.6 billion in Canada, and it is estimated that these costs will quadruple by 2030 due to aging of the population. Osteoporosis fractures are more common than stroke, heart attack and breast cancer combined. Current treatments of osteoporosis are based on drugs but current medications are insufficient to strengthen bones and prevent fractures, while causing multiple undesirable side effects and atypical fractures. There is thus an important medical need for osteoporosis treatments that are more effective than medication, with fewer side effects.

Electro-medicine is a field that exists for many years. Devices using electrical stimulations have been suggested and even approved by medical authorities for heart diseases (e.g., pacemakers, cardiac defibrillators), for bladder and bowel problems (e.g., sacral neuromodulation (SNM)), treatment of pain (transcutaneous electrical nerve stimulation (TENS), regeneration and recovery of neuromusculoskeletal injuries (e.g., electrolysis and electra-stimulation) and targeted fat loss (YourTrimX™ electrical stimulator sold in the past by LumaLife™ (see YourTrimX™ FaceBook™ web page and U.S. Pat. No. 7,933,647)).

Technologies that have been used or tested to improve bone healing include externally applied electrical stimulation (EStim), pulsed electromagnetic field (PEMF) and low-intensity pulsed ultrasound (LIPUS). However, these technologies are different because they do not stimulate bones with an electric signal (e.g. PEMF, LIPUS), and/or because their efficacy is uncertain or because they cause discomfort to patients due to muscle contractions generated by the stimuli and they require high stimulation intensities that are difficult to tolerate (e.g., EStim).

There have been a few reports concerning the use of electrical fields in the amelioration of osteoporosis (Liriani-Galvão APR et al. (2006), Brazilian J Med Biol Res. 39: 1501-1505; Tabrah F et aL (1990), JBone Miner Res. 5(5):437-42; and Giordano Net al. (2001), Curr Therap Res. 62(3):187-93). However, in all these studies, the reported effects on the bone were rather “indirect” since the electrical fields were used to induce muscle contractions (so-called functional electrical stimulation) rather than direct bone stimulation.

Accordingly, there is a need for the promotion of bone growth, the promotion of bone mineralization and/or formation, and/or the promotion of an increase in bone density.

There is also a need for methods and devices for preventing and/or healing bone fractures, particularly methods and devices that do not cause discomfort to patients or do not cause undesirable side effects.

There is particularly a need for a non-invasive treatment of bone diseases by using electrical stimulation requiring only a low-intensity electrical current avoiding muscle contraction.

There is also a need for devices and methods and/or preventing risk of fractures due to osteoporosis for treating osteoporosis, particularly devices and methods that are more effective than existing medications and that do not cause side effects.

The present invention addresses these needs and other needs as it will be apparent from the review of the disclosure and description of the features of the invention hereinafter.

### SUMMARY OF THE INVENTION

According to one aspect, the invention relates to a method for promoting bone growth, comprising directly electrically stimulating bone cells with a low-intensity electrical current avoiding muscle contraction.

According to another aspect, the invention relates to a method for preventing bone loss and/or for healing a bone in a subject, comprising: contacting at least one pair of electrodes on a skin surface over said bone; and providing to the at least one pair of electrodes a low-intensity electrical current avoiding muscle contraction; wherein the electrical current stimulates bone cells.

According to another aspect, the invention relates to a device for electrical stimulation of bone tissues and/or endothelial cells, the device comprising an electrical stimulator configured for providing to the bone tissues a low-intensity electrical current avoiding muscle contraction.

According to another aspect, the invention relates to the use of a device as defined herein, for maintaining bone quality or bone health, for treating osteoporosis, for promoting bone healing, for treating fractures, for treating spinal fusion and/or for spinal cord injury.

According to another aspect, the invention relates to a kit for electrically stimulating bone tissues, comprising: (i) at least one pair of electrodes to be contacted with a skin surface above the bone tissues; and (ii) a device adapted to be operatively connected to the at least one pair of electrodes, the device being configured for providing a low-intensity electrical current to the at least one pair of electrodes.

According to another aspect, the invention relates to a system for monitoring electrical stimulation of bone tissues in a subject, comprising a device as defined herein.

In embodiments the low-intensity electrical current is a biphasic current. In embodiments the low-intensity electrical current has an intensity of about 6.5 mA to about 10.5 mA. In embodiments, the low-intensity electrical current comprises a high frequency (HF) modulated by a low frequency (LF). In embodiments, high frequency is about 800 Hz to about 1000 Hz, and the low frequency is about 1 Hz.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE DRAWINGS

In order for the invention to be readily understood, embodiments of the invention are illustrated by way of example in the accompanying drawings.

FIG. 1A is a diagram illustrating a biphasic electrical current for the electrical stimulation of bones, in accordance with one particular embodiment of the invention;

FIG. 1B is a diagram illustrating a high frequency (HF) current modulated by a low frequency current, in accordance with one particular embodiment of the invention;

FIG. 2A and 2B are pictures of a bottom-side perspective view of a waist belt incorporating an apparatus for the electrical stimulation of bones, in accordance with one embodiment of the invention;

FIG. 3 is a picture of a rectangular-shaped skin pad comprising an anode and a cathode, in accordance with one embodiment of the invention;

FIG. 4A is a picture of a butterfly-shaped skin pad comprising a pair of electrodes, in accordance with one embodiment of the invention;

FIG. 4B is a picture of another type of butterfly-shaped skin pad comprising a pair of electrodes, in accordance with another embodiment of the invention;

FIG. 5A shows a male subject wearing the waist belt of FIG. 2, the subject also wearing on its right hip a skin pad connected to the waist belt, in accordance with one embodiment of the invention;

FIG. 5B shows another embodiment of a waist belt to which is connected the skin pad of FIG. 4B connected to the waist belt, in accordance with another embodiment of the invention;

FIG. 6 is a picture showing an exploded view of a kit in accordance with one embodiment of the invention;

FIG. 7 is a picture showing a waist belt connected to a desktop charger, in accordance with one embodiment of the invention; and

FIG. 8 is a line graph showing positive effect of electrical stimulation on the bone mineral content (BMC)) and bone surface area (BSA) of the legs, in accordance with Example 2.

Further details of the invention and its advantages will be apparent from the detailed description included below.

### DESCRIPTION OF THE PREFERRED EMBODIMENTS

In the following description of the embodiments, references to the accompanying drawings are illustrations of an example by which the invention may be practised. It will be understood that other embodiments may be made without departing from the scope of the invention disclosed. Unless defined otherwise, all technical and scientific terms used herein have the same meaning as commonly understood by one of ordinary skill in the art to which the invention belongs.

### General Overview

The present invention provides, among other things, methods and devices for promoting bone growth by using electrical stimulations.

The present inventors have found that it is possible to positively affect bones, e.g. to promote bone growth, to increase bone mineral content, to increase bone mineral content and/or to increase bone density, by transmitting electrical impulses through the skin of a subject, these electrical impulses consisting of a low-intensity current avoiding muscle contraction.

The methods and devices according to the present invention may find numerous utilities in maintaining bone quality and in promoting bone health. The present invention also possesses numerous benefits, including the fact it can provide a noninvasive treatment of bone diseases, such as osteoporosis. It could also replace, or at least supplement, existing medications which typically have side effects.

### Method for Promoting Bone Growth and Method for Preventing Bone Loss and/or Healing a Bone

One aspect of the invention concerns a method for promoting bone growth, comprising directly electrically stimulating bone cells with a low-intensity electrical current avoiding muscle contraction.

Another related aspect of the invention concerns a method for preventing bone loss and/or for healing a bone in a subject, comprising contacting at least one pair of electrodes on a skin surface (i.e. anode or positive terminal and cathode or negative terminal), and propagating between the two electrodes a low-intensity electrical current avoiding muscle contraction. In embodiments, this low-intensity electric current goes through the skin to reach the bone tissue and stimulates bone cells.

As used herein, “directly electrically stimulating” or “electrically stimulate directly” with reference to electrical stimulation of bone cells or bone tissues, does not refer to the type of current (i.e. direct current (DC) vs alternative current (AC)) but to an electrical stimulation which targets specifically the bone(s) and not other tissues such as muscle, nervous system or skin.

As used herein, the terms “low-intensity electrical current” or “stimulation with a current of low intensity” or similar expression(s) refer to an electrical current that avoids (i.e. does not cause) muscle contraction (e.g. the current is too weak to stimulate the muscle). In embodiments, these terms encompass an electrical current that also avoids stimulation or recruitment of nerves of a subject. In embodiments, the terms refer to a current of about 5 mA to about 11 mA, preferably about 6.5 mA to about 10.5 mA.

In accordance with embodiments, the low-intensity electric current and associated direct stimulation of the bone cells or tissues provides at least one of the following benefits: promotion of bone growth; promotion of bone mineralization (i.e. bone mineral content (BMC)); increasing bone surface area (BSA); promotion of bone density (i.e. ratio of bone mineral content BMC and BSA)); stimulation of osteogenesis via proliferation of H-type vessel endothelial cells; modulation and/or interference in signaling pathways that regulate H-type vessels; promoting release of cytokines and growth factors secreted by vascular endothelial cells: affecting osteocyte interconnections and periosteocytic mineralization.

Such benefit(s) may be measurable for instance in a subject having been subjected to the electrical stimulation compared to a not-stimulated subject. The benefit(s) may also be measured and/or quantified using suitable bone-analysis methods such as dual-energy X-ray absorptiometry (DXA).

The methods according to the present invention may find utilities in maintaining bone quality or bone health. For instance, these methods may be used for preventing bone loss and/or for promoting and/or accelerating healing of the bones of a subject (e.g. healing of bone fractures, healing of a spinal fusion or spinal-cord injury).

As defined herein the term “subject” includes vertebrates and more particularly mammals. The term “subject” includes domestic animals (e.g. cats, dogs, horses, pigs, cows, goats, sheep), rodents (e.g. mice or rats), rabbits, squirrels, bears, primates (e.g., chimpanzees, monkeys, gorillas, and humans), wild animals such as those living in zoos (e.g. lion, tiger, elephant, and the like), and transgenic species thereof. Preferably, the subject is a human (man or woman), more preferably a human patient in need of bone treatment. Even more preferably the mammalian subject is a human patient diagnosed or susceptible to suffer from a bone degenerative disease or a bone loss disease such as osteopenia and osteoporosis. The present invention may possibly also find utilities in treating deterioration of cartilage such as osteoarthritis. In particular embodiments the subject is selected from the group including, but not limited to, post-menopausal women, over-trained athletic women, children with osteogenesis imperfecta, adults of 45 years or more (e.g. male adults of 50 years or more), and astronauts. In embodiments the patient has a bone fracture.

As used herein, the terms “treatment” or “treating” of a subject include direct electrical stimulation of the patient's bone cells with a low-intensity electrical current avoiding muscle contraction, with the purpose of stabilizing, curing, healing, alleviating, relieving, altering, remedying, less worsening, ameliorating, improving, or affecting the bone disease or condition, the symptoms of the bone disease or condition, or the risk of (or susceptibility to) the bone disease or condition. The term “treating” refers to any indication of success in the treatment or amelioration of an injury, pathology or condition, including any objective or subjective parameter such as abatement; remission; lessening of the rate of worsening; lessening severity of the bone disease; stabilization, diminishing of symptoms or making the injury, pathology or condition more tolerable to the subject; slowing in the rate of degeneration or decline; making the final point of degeneration less debilitating; or improving a subject's physical or mental well-being.

Without being bound by any theory, it is suggested that a direct electrical stimulation of bone cells with a low-intensity electrical current in accordance with the present invention affects (e.g. modulate, activate, inhibits) the biochemistry and/or biological activity of the bone tissues. Indeed, the low-intensity electrical current may act directly on the osteoblasts (the cells that form new bone) and/or the osteoclasts (the bone cells that breaks down bone tissue). For instance, the electrical stimulation may involve activation of the osteoblasts causing an increase in osteocalcin (OC).

Accordingly, in embodiments, the methods of the invention stimulate proliferation of osteoblasts and/or they stimulate differentiation of osteoblasts, thereby promoting bone growth. In embodiments, the methods of the invention inhibit the proliferation of osteoclasts, they inhibit differentiation of osteoclasts, they inhibit bone resorption controlled by osteoclasts and/or cause an increase in osteocalcin (OC), thereby promoting bone growth.

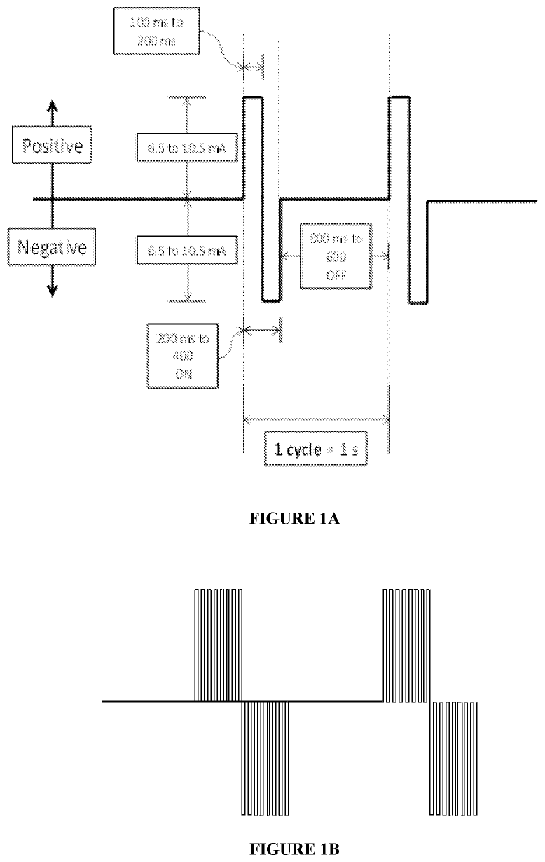

In embodiments, the low-intensity current is a “biphasic current”. As used herein, the term biphasic is a current characterized with an “ON” time when the current is delivered and an “OFF” time when no current is applied. During the “ON” time the current is comprised of both, a positive and a negative phase. During a 1^{st} phase (i.e. positive phase) the ions travel from the anode to the cathode whereas during a 2^{nd} phase (i.e. negative phase) the ions travel from the cathode to anode since the current polarity is reversed. Preferably, the ON time is composed of square waves (positive and negative) of the same duration, the same intensity and, hence, the same charge, one square wave canceling the effect of the other (e.g. FIG. 1A). It was found that a biphasic current is preferable in accordance with the present invention because with such biphasic current there is no accumulation of ions under an electrode, no polarization and, hence, no burning sensation and no burns. In accordance with a preferred embodiment, the electric stimulation comprises high frequency pulses of monophasic current as illustrated in FIG. 1B. Each pulse is monophasic, i.e. the ON time comprises a positive phase only and two consecutive pulses are spaced apart by a period of time during which no current is applied (interstimulus delay).

In embodiments, the low intensity current in accordance with the present invention has an intensity between about 4 mA to about 11 mA, or about 5 mA to about 10.5 mA, or about 6 mA to about 10.5 mA. In one preferred embodiment, the intensity is about 6.5 mA to about 10.5 mA. In embodiments the intensity is about 4 mA, or about 5 mA, or about 6 mA, or about 6.5 mA, or about 7 mA, or about 7.5 mA, or about 8 mA, or about 20 8.5 mA, or about 9 mA, or about 9.5 mA, or about 10 mA, or about 10.5 mA, or about 11 mA,

In embodiments, the low intensity current in accordance with the present invention has a voltage of about 1V to about 50V, or about 5V to about 49V or about by to about 48V. In embodiments the minimal voltage value is adjusted in accordance with the minimum desired current intensity and the maximal voltage value is adjusted in accordance to maximal voltage authorized by regulatory authorities (e.g. about 50V as per FDA regulations for biomedical devices). In embodiments, the low intensity current in accordance with the present invention has a voltage of about 5V or about 7.5V or about 10V, or about 15V, or about 20V, or about 25V, or about 30V, or about 35V, or about 40 V, or about 45 V, or about 46V, or about 47V. In one preferred embodiment, the voltage is adjusted constantly by the device in accordance with the impedance at the electrodes (i.e. resistance) in order to maintain a constant current intensity. Nevertheless, for safety reasons, the device is preferably set to a maximum voltage of 47V. If the impedance increases to a level requiring more than 47V, the device issues a warning signal indicating that usage should be stopped, that the pair of electrodes removed and be replaced by new ones and/or that the skin be cleaned, for allowing the device to go back to a voltage below the maximum of 47V.

In accordance with the present invention, the current is preferably biphasic. Therefore, the total charge of a pulse (composed of a positive and a negative phase) is of 0 microC. However, when considering one phase individually (positive or negative) the charge preferably ranges from about 325 microC to about 2625 microC, depending on the intensity of the current (e.g. from about 6.5 mA to about 10.5 mA) and the duration of a phase (e.g. from about 100 ms for a duty cycle of 20/80 to about 200 ms for a duty cycle of 40/60). In particular embodiments, the low intensity current in accordance with the present invention has a charge of 300 microC, or 325 microC, or 400 microC, or 500 microC, or 600 microC, or 700 microC, or 800 microC, or 900 microC, or 1000 microC, or 1100 microC, or 1500 microC, or 1750 microC, or 2000 microC, or 2100 microC, or 2500 microC, or 2600 microC or or 2625 microC.

In embodiments, the low intensity current in accordance with the present invention has a power which varies according to the voltage and the current intensity. In one particular embodiment the power is from about 0.3055 W to about 0.4935 W for a maximum voltage of 47 V and current intensity ranging from 6.5 mA to 10.5 mA. In embodiments the power is about 0.3 W, or about 0.35 W, or about 0.4 W, or about 0.45 W, or about 0.5 W.

In embodiments, the low intensity current in accordance with the present invention has an energy which varies according to the power of a pulse and the pulse duration. In embodiments the energy is about 0.0153 J to about 0.0987 J for a pulse of power of about 0.3055 W to about 0.4935 W and a duration of about 100 ms for a duty cycle of 20/80, and about 200 ms for a duty cycle of 40/60. In embodiments the energy is about 0.01 J, or about 0.02 J, or about 0.03 J, or about 0.04 J, or about 0.05 J, or about 0.06 J, or about 0.07 J, or about 0.08 J, or about 0.09 J or about 0.1 J.

Current density is defined as the power of the current divided by the surface area of the pair of electrodes or skin pad. Accordingly, the density will vary depending on both of these parameters. In embodiments, the low intensity current in accordance with the present invention has a density from about 1.5 W/cm² to about 3.5 W/cm², e.g., about 1.5 W/cm², or about 2 W/cm², or about 2.5 W/cm², or about 3 W/cm², or about 3.5 W/cm². In one particular embodiment the electrode has a surface area of about 24 inches² (4 inches×6 inches) [about 154.84 cm2 (10.16 cm by 15.24 cm2)] and hence, the current density ranges from 1.97 mW/cm2 to 3.19 mW/cm2 depending upon the power (ranging from 0.3055 W to 0.4935 W or 305.5 mW to 493.5 mW).

In accordance with the present invention, the current may be symmetrical or asymmetrical. In embodiments the current is asymmetrical. As is known, an “asymmetrical current” is a current in which the percentage of ON time and percentage OFF time (also known as “Duty cycle”) is other than 50/50 (i.e. as opposed to a symmetrical current with 50% ON time and 50% OFF time). In embodiments the ON time is shorter than the OFF time, e.g., from about 1/99 to about 49/51, or from about 10/90 to about 45/55, or from about 20/80 to about 40/60. In embodiments, the duty cycle (i.e. ON/OFF) is about 5/95, or about 10/90, or about 15/85, or about 20/80, or about 25/75, or about 30/70, or about 35/65, or about 40/60, or about 45/55.

In embodiments, the asymmetrical current has a low frequency (LF). As defined herein the term “low frequency” or “LF” refers to a cycle of about 0.5 Hz to about 2 Hz, e.g. about 0.5 Hz, or about 1.5 Hz. In one particular embodiment the asymmetrical current has a low frequency (LF) of about 1 Hz.

In embodiments, and as illustrated in FIG. 1B, the low intensity current in accordance with the present invention preferably combines two different ranges of frequencies: a high frequency (HF) modulated by a low frequency (LF). In this type of current a LF impulse is composed of several HF impulses instead of being of a continuous current. In accordance with the present invention using two different ranges of frequencies may be advantageous since it may be more comfortable to subjects because the HF is less efficient in “recruiting” sensitive nerves. It may also drain less energy on the stimulator, thereby sparing the battery life and/or reducing the size of the battery required.

The combination of LF and HF in one current thus brings together the advantages of both types of currents: use of LF which is a current more efficient for the recruitment of physiological units (i.e., cells) and the use of HF which may provide a more comfortable experience and an extended battery life.

In embodiments, the HF impulses are from about 500 Hz to about 5000 Hz or about 600 Hz to about 2500 Hz, or about 700 Hz to about 1500 Hz. In one particular embodiment the HF impulses are from 800 Hz to about 1000 Hz, e.g., about 800 Hz, or about 850 Hz, or about 900 Hz, or about 950 Hz, or about 1000 Hz. In embodiments, the low intensity current in accordance with the present invention comprises two types of asymmetrical biphasic currents in a range of intensity and duty cycle defined as follows: (1) Low Frequency with: Frequency=1 Hz; Duty Cycle ranging from 20/80 to 40/60; Intensity ranging from 6.5 mA to 10.5 mA; and (2) High Frequency modulated by Low Frequency with: LF=1 Hz; HF monophasic symmetrical current ranging from 800 Hz to 1000 Hz; LF Duty Cycle ranging from 20/80 to 40/60; Intensity ranging from 6.5 mA to 10.5 mA.

In embodiments, the low-intensity current is selected from the height (8) following currents:
1. 1 H LF, 6.5 mA, 20/80 duty cycle;
2. 1 H LF, 10.5 mA, 20/80 duty cycle;
3. 1 H LF, 6.5 mA, 40/60 duty cycle;
4. 1 H LF, 10.5 mA, 40/60 duty cycle;
5. 800 Hz HF modulated by 1 Hz LF, 6.5 mA, 20/80 duty cycle;
6. 800 Hz HF modulated by 1 H LF, 10.5 mA, 20/80 duty cycle;
7. 1000 Hz HF modulated by 1 H LF, 6.5 mA, 20/80 duty cycle; and
8. 1000 Hz HF modulated by 1 H LF, 10.5 mA, 20/80 duty cycle.

In one particular embodiment, the low-intensity current is in accordance with the following parameters: 1 Hz (400 ms ON (200 ms positive (up), 200 ms negative (down), and 600 ms OFF with neutral voltage).

In one particular embodiment, the low-intensity current is in accordance with the following parameters:
same voltage for each electrode, i.e. about 7.74 rnV (minimum about
0.55 mV, maximum about 26.59 mV), one electrode positive, the other in negative;
a medium resistance of about 16818 kΩ (minimum about 14498 kΩ), maximum about 19221 kΩ); and
an electrical current of about 0.0046 mA (minimum about 2.86*10⁻⁵ mA, maximum about 0.0183 mA).

In another particular embodiment, the low-intensity current is in accordance with the following parameters:
about +/−0.38 mV (minimum about 0.0027 mV, maximum about 608.89 mV) on each electrode (one positive, the other in negative);
an electrical current of about 458*104 mA (minimum about 3,735*10⁻⁵ mA, maximum about 0.063 mA).

Tables 1 to 5 hereinafter provide additional electrical current characteristics for selected symmetrical currents and asymmetrical currents in accordance with the present invention. An example of calculation of electrical current characteristics is provided in Example 1. Those skilled in the art can readily make similar calculation for other currents encompassed by the present invention.

TABLE 1
Electrical current characteristics for symmetrical currents
Symmetrical Currents

| | Frequency | | | | | |
|---|---|---|---|---|---|---|
| | | | HF = 800 Hz | | HF = 1000 Hz | |
| | LF = 1 Hz | | LF = 1 Hz | | LF = 1 Hz | |
| | Intensity | | | | | |
| | 6.5 mA | 10.5 mA | 6.5 mA | 10.5 mA | 6.5 mA | 10.5 mA |
| | Duty Cycle | | | | | |
| | 50/50 | | | | | |
| Total charge (microC) | 0 | 0 | 0 | 0 | 0 | 0 |
| Charge (microC) | 1625 | 2625 | 812.5 | 1312.5 | 812.5 | 1312.5 |
| Power (W) | 0.3055 | 0.4935 | 0.3055 | 0.4935 | 0.3055 | 0.4935 |
| Energy (J) | 0.03055 | 0.04935 | 0.03055 | 0.04935 | 0.03055 | 0.04935 |
| Density (W/cm²) | 1.97 | 3.19 | 1.97 | 3.19 | 1.97 | 3.19 |

TABLE 2
Electrical current characteristics for asymmetrical LF (1 Hz) currents
Asymmetrical Low Frequency (1 Hz) Currents

| Intensity | 6.5 mA | | | | |
|---|---|---|---|---|---|
| Duty Cycle | 20/80 | 25/75 | 30/70 | 35/65 | 40/60 |
| Total charge (microC) | 0 | 0 | 0 | 0 | 0 |
| Charge (microC) | 650 | 812.5 | 975 | 1137.5 | 1300 |
| Power (W) | 0.3055 | 0.3055 | 0.3055 | 0.3055 | 0.3055 |
| Energy (J) | 0.03055 | 0.0611 | 0.0611 | 0.0611 | 0.0611 |
| Density (W/cm²) | 1.97 | 1.97 | 1.97 | 1.97 | 1.97 |

| Intensity | 10.5 mA | | | | |
|---|---|---|---|---|---|
| Duty Cycle | 20/80 | 25/75 | 30/70 | 35/65 | 40/60 |
| Total charge (microC) | 0 | 0 | 0 | 0 | 0 |
| Charge (microC) | 1050 | 1312.5 | 1575 | 1837.5 | 2100 |
| Power (W) | 0.4935 | 0.4935 | 0.4935 | 0.4935 | 0.4935 |
| Energy (J) | 0.04935 | 0.0987 | 0.0987 | 0.0987 | 0.0987 |
| Density (W/cm²) | 3.19 | 3.19 | 3.19 | 3.19 | 3.19 |

TABLE 3
Electrical current characteristics for Asymmetrical High Frequency
(800 Hz) modulated Low Frequency (1 Hz) currents
Asymmetrical High Frequency (800 Hz) modulated Low Frequency (1 Hz)

| Intensity | 6.5 mA | | | | |
|---|---|---|---|---|---|
| Duty Cycle | 20/80 | 25/75 | 30/70 | 35/65 | 40/60 |
| Total charge (microC) | 0 | 0 | 0 | 0 | 0 |
| Charge (microC) | 325 | 406.25 | 487.5 | 568.75 | 650 |
| Power (W) | 0.3055 | 0.3055 | 0.3055 | 0.3055 | 0.3055 |
| Energy (J) | 0.015275 | 0.0611 | 0.0611 | 0.0611 | 0.03055 |
| Density (W/cm²) | 1.97 | 1.97 | 1.97 | 1.97 | 1.97 |

| Intensity | 10.5 mA | | | | |
|---|---|---|---|---|---|
| Duty Cycle | 20/80 | 25/75 | 30/70 | 35/65 | 40/60 |
| Total charge (microC) | 0 | 0 | 0 | 0 | 0 |
| Charge (microC) | 525 | 656.25 | 787.5 | 918.75 | 1050 |
| Power (W) | 0.4935 | 0.4935 | 0.4935 | 0.4935 | 0.4935 |
| Energy (J) | 0.024675 | 0.0987 | 0.0987 | 0.0987 | 0.04935 |
| Density (W/cm²) | 3.19 | 3.19 | 3.19 | 3.19 | 3.19 |

TABLE 4
Electrical current characteristics for Asymmetrical High Frequency
(100 Hz) modulated Low Frequency (1 Hz) currents
Asymmetrical High Frequency (1000 Hz) modulated Low Frequency (1 Hz)

| Intensity | 6.5 mA | | | | |
|---|---|---|---|---|---|
| Duty Cycle | 20/80 | 25/75 | 30/70 | 35/65 | 40/60 |
| Total charge (microC) | 0 | 0 | 0 | 0 | 0 |
| Charge (microC) | 325 | 406.25 | 487.5 | 568.75 | 650 |
| Power (W) | 0.3055 | 0.3055 | 0.3055 | 0.3055 | 0.3055 |
| Energy (J) | 0.015275 | 0.0611 | 0.0611 | 0.0611 | 0.03055 |
| Density (W/cm²) | 1.97 | 1.97 | 1.97 | 1.97 | 1.97 |

| Intensity | 10.5 mA | | | | |
|---|---|---|---|---|---|
| Duty Cycle | 20/80 | 25/75 | 30/70 | 35/65 | 40/60 |
| Total charge (microC) | 0 | 0 | 0 | 0 | 0 |
| Charge (microC) | 525 | 656.25 | 787.5 | 918.75 | 1050 |
| Power (W) | 0.4935 | 0.4935 | 0.4935 | 0.4935 | 0.4935 |
| Energy (J) | 0.024675 | 0.0987 | 0.0987 | 0.0987 | 0.04935 |
| Density (W/cm²) | 3.19 | 3.19 | 3.19 | 3.19 | 3.19 |

TABLE 5
Electrical current characteristics for electrical stimulation
at electrodes contact on the skin at the level of the hips^{#}

| | | Electric potential on the | | Approximated electric potential | |
|---|---|---|---|---|---|
| Electric current on | | skin* | | at periosteum* | |
| the skin | | (min = thin, max = fat) | | (min = fat, max = thin) | |
| 6.5 | mA | 5.75 to 6.24 | V | 7.6 to 9.6 | mV |
| 7.0 | mA | 6.17 to 6.74 | V | 8.2 to 10.3 | mV |
| 7.5 | mA | 6.65 to 7.23 | V | 8.8 to 11.1 | mV |
| 8.0 | mA | 7.07 to 7.72 | V | 9.4 to 11.8 | mV |
| 8.5 | mA | 7.49 to 8.22 | V | 10.0 to 12.5 | mV |
| 9.0 | mA | 7.91 to 8.71 | V | 10.6 to 13.2 | mV |
| 9.5 | mA | 8.39 to 9.20 | V | 11.2 to 14.0 | mV |
| 10.0 | mA | 8.80 to 9.70 | V | 11.8 to 14.7 | mV |

| | | | | |
|---|---|---|---|---|
| About 6.5 mA to | 10 to 20 | V | 12.2 to 33.39 | mV |
| about 10 mA | 20 to 30 | V | 24.34 to 50.09 | mV |
| | 30 to 40 | V | 36.51 to 66.78 | mV |

*Calculations made using a voltage of 1 Hz (400 ms ON (200 ms positive (up), 200 ms negative (down), and 600 ms OFF with neutral voltage).
*Thin and fat refers to the body type (e.g. slim shape or fat physique).

In embodiments, and according to Table 5, a current of about 6.5 mA to about 10 mA or voltage of about 5 V to about 40 V applied on the skin, would open ionic channels involved in bone formation. In embodiments, and electric current of about 6.5 mA to about 10 mA on the skin at the level of the hips, after passing through every layer of all body tissues (e.g. skin layers, fat, muscle, tendon, etc.), would cause an electric potential change on bone cells' membrane of about 7.6 mV to about 14.7 mV, which is enough to open calcium voltage dependent ionic channels involved in bone formation.

In embodiments, and according to Table 5, an electric potential of about 10 V to about 40 V on the skin at the level of the hips, after passing through every layer of all body tissues, would cause an electric potential change on bone cells' membrane of about 12 mV to about 70 mV, which is also enough to open calcium voltage dependent ionic channels involved in bone formation.

In embodiments, and according to Table 5, an electric potential on the skin at the level of the hips of about 5 V to about 40 V would mean an electric potential change on bone cells' membrane of about 7 mV to about 70 mV, which also enough to open ionic channels.

In embodiments, the low-intensity electrical current according to the present invention comprises and/or provide one or more of the following properties:
biphasic;
alternate current;
square wave;
low frequency from about 1 Hz
high frequency pulses from about 800 Hz to 1000 Hz;
high frequency pulses on time from about 0.500 ms to about 0.625 ms and off time from about 0.500 ms to about 0.625 ms;
amperage from about 6.5 mA to about 10.5 mA;
voltage from about 5 V to about 50 V (e.g. about 10 V to about 47 V);
total charge of a pulse of 0 coulomb;
total charge of about 325 microcoulomb to about 2625 microcoulomb;
energy of about 0.0153 joule to about 0.0987 joule;
a density of about 1.5 W/cm2 to about 3.5 W/cm2;
an electric potential between about 7.5 mV to about 67 mV at the level of the membrane of bone cells;
is defined in accordance with the representation of FIG. 1A and/or FIG. 1B

In embodiments, the low-intensity electrical current is other than a current which is being used, or that may be used, for existing transcutaneous electro-medicine devices. In embodiments, the low-intensity electrical current according to the present invention is other than an electromagnetic current, other than ultrasound, other than electrolysis, other than sacral neuromodulation (SNM), and other than transcutaneous electrical nerve stimulation (TENS).

Table 6 hereinafter provides an overview of a preferred embodiment of the low-intensity current in accordance with the present invention compared with other existing electro-medicine devices. In embodiments, the low-intensity current of the invention is other than any of these existing current.

TABLE 6
Low-intensity current of present invention compared to current in other devices
Transcutaneous Electrical Stimulation

| | | Parameters | | | | | | |
|---|---|---|---|---|---|---|---|---|
| | | Current | | Frequency | | | | |
| | | Amperage (mA) | | Hertz (Hz) | | Active time per sec. | | |
| Category | Application(s) | min | max | min | max | ON-min | OFF-max | Mode |
| Rehabilitation | Physiotherapy, | 0 | 99.5 | 0 | 120 | 0 | 48 ms | Alternative |
| (SNM) [symbol] | muscle | | | | | | | |
| | rehabilitation | | | | | | | |
| Pain | Nerve | 0 | 80 | 2 | 120 | 80 μs | 30 ms | Alternative |
| management | stimulation to | | | | | | | |
| (TENS) [symbol] | reduce pain | | | | | | | |
| Cardiac | Reanimation | 400 | 100 000 | 2000 | 8000 | 1 ms | 50 ms | Impulse train |
| defibrillators [symbol] | | | | | | | | |
| Fat Tissue: | Upolysis | 6 | 6 | 1 | 1 | 500 ms | 500 ms | Symmetrical |
| Adipotronics [symbol] | stimulation | (constant | (constant | | | | | alternating |
| | | current) | current) | | | | | current |
| Present | Increase in bone | 6.5 | 10.5 | 1 | 1 | 200 ms to | 600 ms to | Symmetrical |
| invention | surface area, | (constant | (constant | | | 400 ms | 800 ms | alternating |
| | bone mineral | current) | current) | | | | | current |
| | content and | | | | | | | |
| | bone density | | | | | | | |

| | Parameters | | | | | | |
|---|---|---|---|---|---|---|---|
| | Voltage | | Micro-Coulomb | | Joules | | |
| | V | | Total Charge | | J | | |
| Category | min | max | min | max | min | max | Remarks |
| Rehabilitation | 0 V | 99.5 V | 0 | 4776 | 0 | .48 | Muscle |
| (SNM) [symbol] | | | | | | | Contractions |
| Pain | 0 V | 110 V | 0 | 21 [symbol] | 0 | .002 | Muscle |
| management | | | | | | | Contractions |
| (TENS) [symbol] | | | | | | | |
| Cardiac | 20 V | 5000 V | 5000 | 120000 | 0.1 | 600 | Heart only |
| defibrillators [symbol] | | | | | | | |
| Fat Tissue: | 10 V | 47 V | 3000 | 3000 | .03 | .141 | Slight |
| Adipotronics [symbol] | | | | | | | muscle |
| | | | | | | | contractions |
| Present | 10 V | 47 V | 1300 | 4200 | 0.013 | 0.197 | Without |
| invention | | | | | | | muscle |
| | | | | | | | contractions |

¹Cefar Rehab ×2: (manual available on the website of VitalityDepot.ca: https://vitalitydepot.ca/content/cefar_primo_pro_eng.pdf)
²TENS: (manual available on the website of VQOrthocare.com: https://www.vqorthocare.com/download/electrotherapy/TENS7000Manual.pdf)
³Cardiac defibrillators: (manual available on the website of FlukeBiomedical.com: https://www.flukebiomedical.com/sites/default/files/7000dp_umeng0100.pdf) [symbol]
indicates data missing or illegible when filed

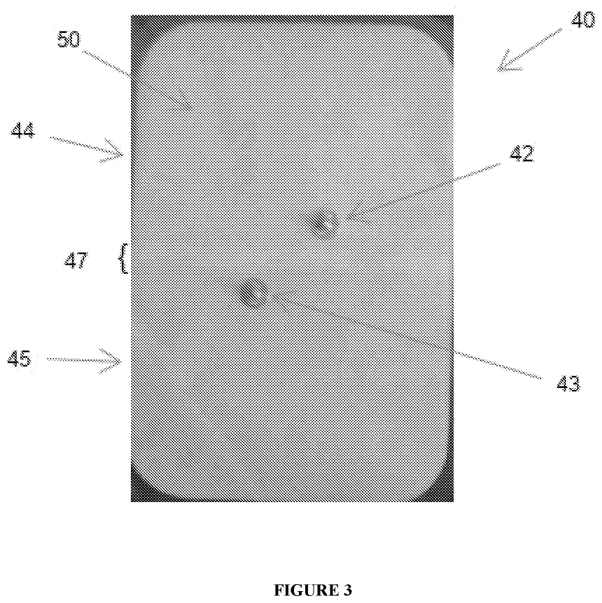

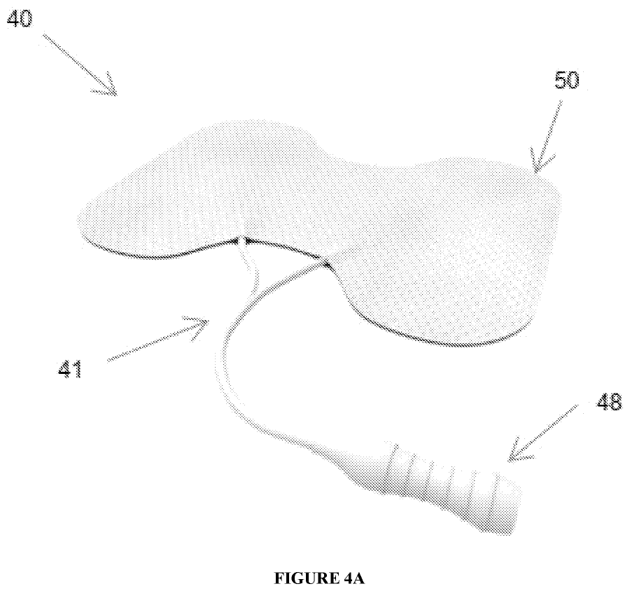

In embodiments, the pair of electrodes are incorporated into a skin pad, as illustrated in FIGS. 3 to 6 and described hereinafter.

Preferably, the number electrodes pair or number of skin ped(s) and their positioning may be adjusted to maximize efficacy, particularly to facilitate and/or to maximize propagation of the current through the skin and muscles, in order to effectivelyreach the bone tissues. Accordingly, the number and positioning may be adjusted inaccordance to parameters such as the bone surface to be treated, the bone density priorto treatment, the muscular or adipose content above the zone to be treated, etc.

In embodiments, the pair of electrodes or skin pad are (is) positioned in a manner that facilitates and/or maximizes propagation of the current through the skin and muscles, in order to effectively reach the bone tissues. In embodiments, a plurality of a pair of electrodes or skin pads surround the bone to be treated and/or a plurality of a pair of electrodes or pads extend longitudinally along the bone requiring treatment. In one particular embodiment the bone to be treated is the hip and one pair of electrodes or one skin pad is positioned each side of the subject. In one particular embodiment the bone to be treated is the wrist and one pair of electrodes or one skin pad is positioned on the wrist of the subject. In one particular embodiment the bone to be treated is the arm and one pair of electrodes or one skin pad is positioned on the arm of the subject. In one particular embodiment the bone to be treated is the spine and one pair of electrodes or one skin pad is positioned on the spine of the subject. In one particular embodiment the bone to be treated is the leg and one pair of electrodes or one skin pad is positioned on the leg of the subject. It is also envisionable to positioned more than one pair of electrodes or more than one skin pad at different locations (e.g. simultaneously on both legs or at multiple location along the spine). A single pair of pair of electrodes or skin pad may also be moved from one region to another in a sequential fashion (e.g. one wrist to the other, from the cervical spine, to thoracic spine to the lumbar spine, etc.). The positioning of the electrodes or skin pad may also be selected in accordance with recommendations of the health regular authorities (e.g. to avoid any risk of current going through the heart). It is within the skills of those in the art or the skills of a physician to identify or suggest a proper treatment intervention for achieving a desired bone growth or bone treatment in accordance with the invention, including positioning and number of electrode(s), stimulus parameters, application techniques, treatment schedules, etc.

In certain embodiments, electric stimulation is carried out according to more than one of the following treatment plans: about 15 min to about 2 h per day; or about 1 to 2 times per day; or about I to 7 times per week; or for a period of I week to about 52 weeks; or for regular sessions along as needed.

The methods of the present invention may also be used in combination with already approved therapies (e.g. drugs or electro-medicine) and/or in combination with training exercise(s). Bisphosphonates are an example of currently approved drug for treating bone conditions. In embodiments the present invention is used in combination with a bisphosphonate medicine, including but not limited to alendronate (e.g. Fosamax®, Binosto®), ibandronate (e.g., Boniva®), risedronate (e.g., Actonel®, Atelvia™) and zoledronic acid (e.g., Reclast™).

In embodiments the present invention is used in combination with medication for prevention and/or treatment of osteopororis drugs such as romosozumab, romosozumab-aqqg (Evenity®). rabxfene (Evsta®), bazedoxifene (Conbza™ Duavee™), teriparatide, ahaloparatde, denasumab, romosozumab, menopausal hormone therapy (MHT), etc. Whenever necessary, a physician may be involved for advising regarding use of drug(s), course of treatment, side effects, etc.

### Low-Intensity Electric Current Stimulation of Osteogenesis Via H-Vessels

Another aspect of the invention concerns the use of low-intensity electrical current for stimulating osteogenesis via H-vessels. Particularly, the device and methods of the present invention may be used to impact on the microvascular structure of bones, for instance by stimulating osteogenesis via the proliferation of H-type vessel endothelial cells, i.e. capillaries CD31hi/Emcnhi also called H-type vessels.

Low-intensity electrical current in accordance with the present invention may also be useful in modulating and/or interfering in signaling pathways that regulate H-type vessels and how they modulate osteogenesis. Known regulatory factors that may be impacted by low-intensity electrical current include, but are not limited to, platelet-derived growth factor BB (PDGF-BB), factor (SLIT3), hypoxia-inducible factor 1-alpha (HIF-1a), Notch and vascular endothelial growth factor (VEGF).

In a related aspect, low-intensity electrical current and device in accordance with the present invention may also be useful in promoting the release of cytokines and growth factors secreted by vascular endothelial cells stimulated in favor of bone formation. The low-intensity electrical current and device in accordance with the present invention may also be useful in affecting osteocyte interconnections and peri-osteocytic mineralization.

In embodiments the low-intensity electrical current and device in accordance with the present invention provides an electric potential that is sufficient to open calcium voltage dependent ionic channels involved in bone formation. In embodiments the lowintensity electrical current and device in accordance with the present invention provides an electric potential of about 7.5 mV to about 67 mM at the level of the membrane of bone cells (e.g., about 7.5 mV to about 15 mV, or about 12 mV to about 34 mV, or about 24 mV to about 50 mV, about 36 mV to about 67 mV, about 12 mV to about 67 mV).

### Device for Electrical Stimulation of Bone Tissues

According to another important aspect, the invention relates to a device for electrical stimulation of bone tissues, this device being configured to provide to the bone tissues to be treated a low-intensity electrical current avoiding (i.e. not causing) muscle contraction. Preferably, the device is configured to electrically stimulate directly bone cells (i.e. total absence of stimulation or no measurable stimulation of the muscle cells). In embodiments, the low-intensity electrical current is as defined hereinbefore.

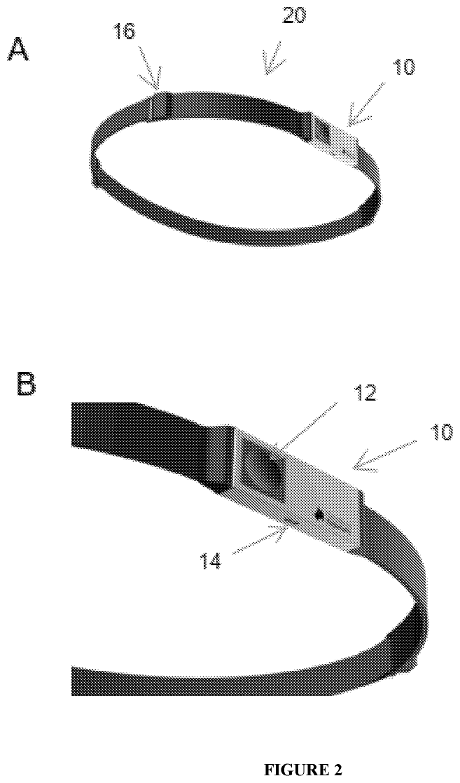

FIGS. 2 to 7 illustrate embodiments of a device 10 in according with the present invention. In these embodiments the device 10 is part of a waist belt 20 to be attached above the hip of a subject. The device 10 comprises an ON/OFF button 12, an internal electrical stimulator, a battery (e.g. a built-in battery) and at least one connector 16 adapted to be operatively connected to the at least one pair of electrodes 40. Preferably, the battery is a rechargeable battery (e.g. a Li-ion battery) and the device is provided with a port 14 (e.g. a mini port for connection such as a mini-USB cable for recharging the battery).

The device 10 may also comprise a memory card (e.g. for storing a user data or stimulation protocols), and wireless functionalities for connection with a mobile device or network (e.g. Bluetooth™, Wi-Fi, RF, 3G, 4G, 5G). The device may also comprise one or more lights (e.g., LED lights for indicating when the device is ON and/or for indicating battery level or charging of the battery and/or for indicating a malfunction (incorrect connection of the wire(s) or electrodes, conductance problems, etc.). Likewise, the device may also piezoelectric sounder(s) that could generate sound(s) when the device is turned ON, when the battery is low, when there is a problem with the connection of the electrodes, etc. The device could also comprise and/or coupled to patient monitoring probes or sensors, including but not limited to, a heart rate monitor, a blood oxygen monitor, an electrocardiogram, a sleep monitor, etc. Typically, the device will further comprise a printed circuit board (PCB)) for mechanically supporting and electrically connecting all the electrical or electronic components defined hereinabove.

Accordingly, the device could be configured to offer real-time monitoring of patients. For instance, the device to be configured to continuously quantify and accurately measure its effects and impacts on users and adjust the provided electric stimulation and/or treatment plan accordingly. The measurements of the device could also be transmitted (automatically or by the user, e.g. via a 5G network) to a specialist who could adjust remotely the patient's stimulation treatment in accordance to the patients situation. Therefore, the present invention also encompasses a system for real-time monitoring and adjustment of electrical stimulation of bone tissues, the system comprising a bone simulation device as defined herein.

The device of the present invention is adapted to be operatively connected to at least one pair of electrodes for providing a low-intensity electrical current thereto. Preferably, the device is adapted to provide two separate electrical stimulation channels such that it is possible to provide low-intensity electrical current distinctively to two separate pairs of electrodes (e.g. one pair positioned on each hip or on each leg). As mentioned hereinbefore, multiple electrode layouts can be envisioned and it may also be envisioned to provide the device with more than two electrical stimulation channels in order to stimulate various regions of the body simultaneously (e.g., hips, legs, back, wrist, arms, spine, etc.).

In embodiments, the device 10 is provided with at least one pair of electrodes 40 to be contacted with a skin surface over the bone(s) to be treated. As illustrated in FIGS. 3 to 6, in embodiments the pair of electrodes 40 comprises an anode 42 (i.e. positive terminal) and a cathode 43 (i.e. negative terminal). The electrodes 40 comprises an electrical wire 41 and a connector 48 for connecting with the device 10 and transporting the electrical current from the device 10 to the anode 42 and cathode 43. It may also be envisioned to use wireless electrodes.

Preferably, the electrodes are incorporated into a skin pad 50 to be applied on the skin of a subject. In embodiments, the skin pad 50 comprises a positive area 44 (i.e. anode 42) and a negative area 45 (i.e. cathode 43) that are separated by a neutral middle section 47 (e.g., FIG. 3). In embodiments, the positive and negative areas of the skin pad both comprise a conductive gel for better electric conductivity.

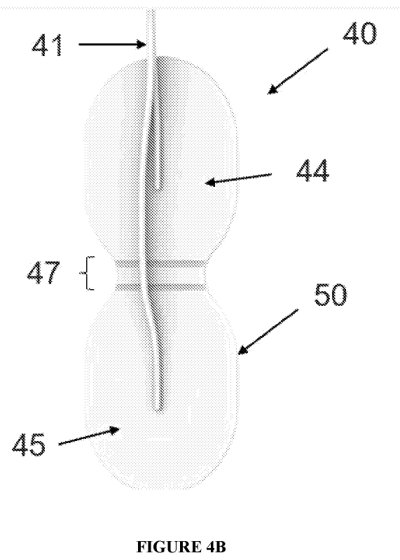

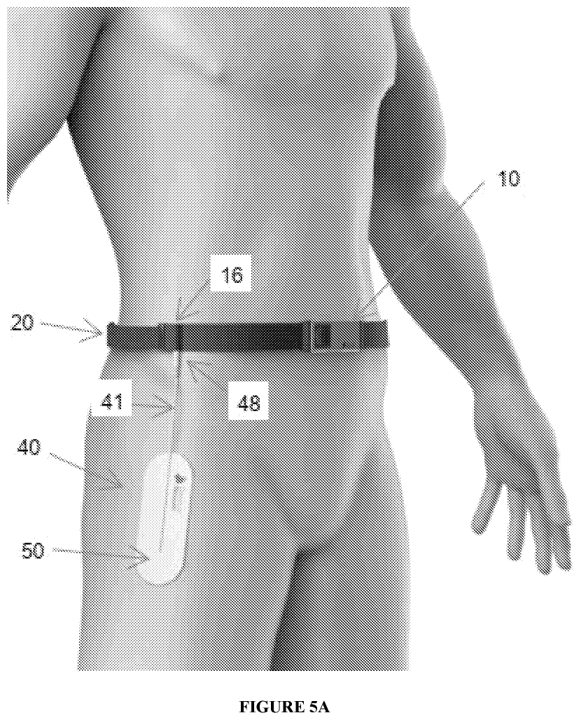

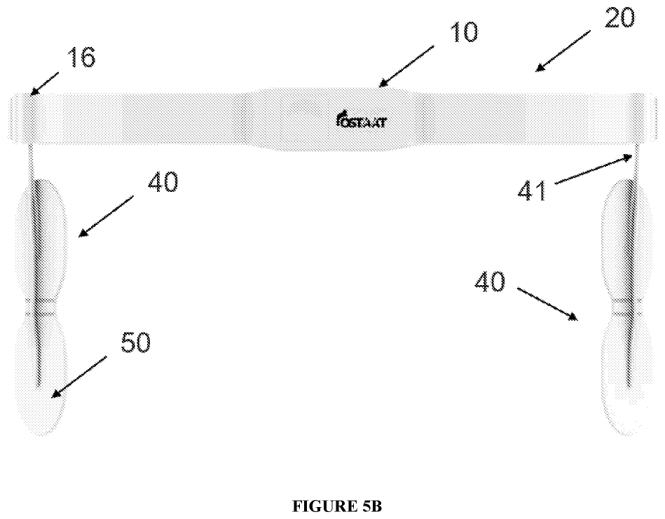

The skin pad can also comprise an adhesive surface covered by a peel-off sheet, for better adherence to the skin of the subject. The skin pad can take any desired shape, including but not limited to a rectangular shape (e.g., FIG. 3), a butterfly shape (e.g., FIGS. 4A and 4B), a cylindrical shape (e.g., FIG. 5), a circle shape, an oval shape, a square shape, a triangular shape, etc. The skin pad 50 can take any desired size, for instance a size adapted to a desired use the bone(s) to be treated (e.g. wrist vs hip), the patient's physiognomy (e.g. small vs tall), etc. In embodiments, the skin pad is particularly adapted for the hip and legs and has a size of about 10 cm×16 cm (about 4 inches×6 inches). In other embodiments, the skin pad is particularly adapted for the wrist and has a size of about 5 cm×5 cm (about 2 inches×2 inches). In other embodiments, the skin pad is particularly adapted for the forearms and has a size of about 5 cm×7.5 cm (about 2 inches×3 inches).

In embodiments, the electrodes are disposable and recyclable. In embodiments, the electrodes are made of a material which facilitates and/or maximizes propagation of the current through the skin and muscles. For instance, the electrodes may be made of carbon, gold, silver, copper, bronze and any other suitable material able to circulate an electric current. In the illustrated embodiments, the pair of electrodes incorporated into the skin pad are made of carbon.

It is conceivable to incorporate the electrodes within a garment and/or within an electrically conductive fabric. Examples of garments include, but are not limited to, a shirt, a sleeveless shirt, a vest, a long pant, a short pant, a sock, a glove, an elbow sleeve, a back belt, a brace, a wrist bracelet, etc. Examples of conductive fabrics which can conduct electricity include, but are not limited to, those comprising a non-conductive or less conductive substrate, which is then either coated or embedded with electrically conductive elements such as carbon, nickel, copper, gold, silver, titanium or Poly(3,4-ethylenedioxythiophene) (PEDOT).

In embodiments, the device according to the present invention comprises one or more of the following properties:
certified in accordance with ISO 13485—PAUMM (MDSAP) global harmonization;
certified in accordance with ISO 14971;
certified in accordance with directive 2011/65/UE: ROHs (Resistance of Hazardous Substance)
certified in accordance with directive UE 217/745 (medical devices)
certified in accordance with the norms IEC/EN 60601-1-6/1EC62366, IEC62 304;
certified in accordance with CSA and CE;
medical device of Class II in Canada;
medical device of Class IIa in Europe;
medical device of Class II in United States.

### Kit for Electrically Stimulating of Bone Tissues

Another aspect of the invention concerns a kit for the electrical stimulation of bone tissues. In one embodiment, the kit comprises: (i) at least one pair of electrodes to be contacted with a skin surface above the bone tissues; and (ii) a device adapted to be operatively connected to said at least one pair of electrodes, the device being configured for providing a low-intensity electrical current to the at least one pair of electrodes.

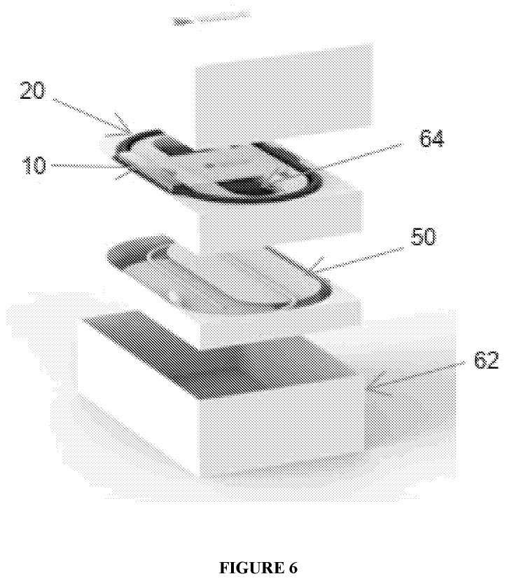

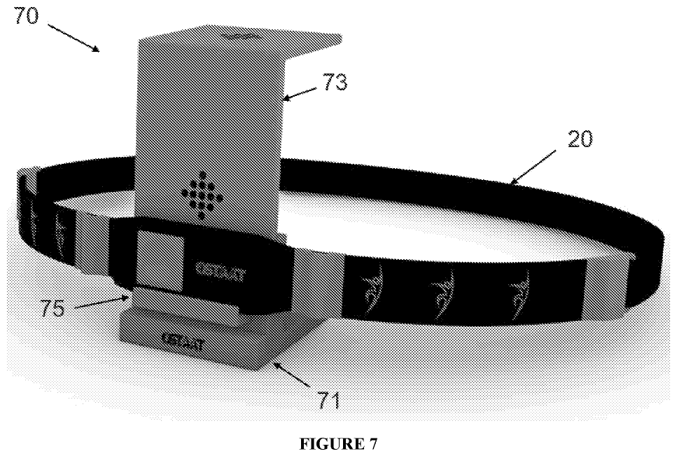

In one embodiment illustrated in FIG. 6, the kit 60 comprises the device for stimulating the bone tissues, the device being integrated within a waist belt 20, a plurality of skin pads 50 each incorporating a pair of electrodes, a box 62 and a mini-USB cable 64 (i.e. for charging the device). Preferably, the kit comprises 2 to 10 pairs of skin pads. The kit may also comprises a desktop charger or holder 70 as illustrated in FIG. 7.

In embodiments, the kit further comprises a transportation bag and a pamphlet with instruction.

In embodiments, the kit further comprises a garment for receiving said at least one pair of electrodes and/or for receiving a pad comprising the electrodes. Examples of garments include, but are not limited to, a shirt, a sleeveless shirt, a vest, a long pant, a short pant, a sock, a glove, an elbow sleeve, a back belt, a brace, a wrist bracelet, etc.

Advantageously, the waist belt 20 as described herein may be connected to a desktop charger or desktop holder 70 as illustrated in FIG. 7. In the illustrated embodiment the desktop charger 70 comprises an horizontal base plate 71 and a vertical wall 73. The vertical wall 73 is provided with a stand 75 for holding the waist belt 20 on the charger 70. In embodiments the vertical wall 73 and/or the stand 75 are provided with an electric connector and/or an induction wireless charger for charging the belt 20.

Those skilled in the art will recognize, or be able to ascertain, using no more than routine experimentation, numerous equivalents to the specific procedures, embodiments, claims, and examples described herein. Such equivalents are considered to be within the scope of this invention, and covered by the claims appended hereto. The invention is further illustrated by the following example, which should not be construed as further or specifically limiting.

### EXAMPLES

### Example 1: Calculations of Electrical Current Characteristics

The following provides an example of calculation of electrical current characteristics for the following current:
Intensity (constant current)=6.5 mA
Frequency (constant)=1 Hz
Symmetrical Biphasic Current:
   20/80 duty cycle
   Impulse duration (ON time)=200 ms
   An impulse is composed of 2 symmetrical phases of 100 ms duration
   OFF time=800 ms
All current characteristics are based on 1 cycle (i.e., I s).

Charge
Charge in Coulombs (C)=A×s (current in amp×time in s)
Charge for I phase: a positive and a negative phase during the ON time
   0.0065 A×0.10 s=0.00065 C (for a phase)
Charge per phase=650 μC
Total Charge per ON time=OpC (0 μC (650 μC−650 μC) one phase balancing the other.

Power
Power in Watts (VV)=Tension (V)×Intensity (A)
Maximal tension=47V
   Max and constant intensity=6.5 mA or 0.0065 A
   Power=47V×0.0065 A=0.3055 W
Maximum Power=0.3055 W

Energy
Energy in Joules (J)
   J=Power (W)×time(s)
   Max Power=0.3055 W
   Phase Duration=0.10 s
   Energy per Phase=0.3055 W×0.10 s
Max Energy=0.03055 J

Current density
Current density=Power (\A per unit of area of application of current (cm²), i.e.
W/cm² or W·cm⁻² (international scientific notation)
   Max Power=0.3055 W
   For a standard electrode 10.16 cm×15.24 cm (4″×6″)
      Surface=10.16 cm×15.24 cm=154.84*cm²
      Current Density=0.3055 W/154.84 cm²=0.00197 W.cm⁻²
Current Density=1.97 mW·cm⁻² (for a 10.16 cm×15.24 cm (4″×6″) electrode)

Summary
Intensity (constant current)=6.5 mA
Frequency (constant)=1 Hz
Symmetrical Biphasic Current
   Impulse duration=200 ms
      An impulse is composed of 2 phases of 100 ms duration
      Duty cycle= 20/80
Total Charge=OC
Maximum Power=0.3055 W
Maximum Energy=0.03055 J
Current Density=1.97 mW/cm² (for a 10.16 cm×15.24 cm (4″×6″) electrode)

### Example 2: Electrical Bone Stimulation in Women

The following study was carried out to demonstrate efficacy in women of a device and method in accordance with the present invention.

### METHODS

### Subjects

A total of 48 women aged between 18 and 45 years old were recruited to participate in this study. They were divided in two groups: a stimulation group (n=24) and a placebo group (n=24). Many of the participants were unable to complete all aspects of the study. At the end of the study, complete data were obtained from 14 women in the stimulation group and 16 women in the placebo group. Physical characteristics of the final sample are shown in Table 6. Groups did not differ significantly on any of these characteristics.

TABLE 6
Physical Characteristics of participants ¹

| | Stimulated | Not Stimulated |
|---|---|---|
| Group: | (N = 14) | (N = 16) |
| Age (y) | 36.36 (5.53) | 33.88 (5.57) |
| Stature (m) | 1.626 (0.084) | 1.634 (0.059) |
| Weight (kg) | 76.94 (15.11) | 81.22 (13.87) |
| Body Mass Index (kg · m⁻²) | 28.88 (3.74) | 30.30 (3.98) |
| % Body Fat from DXA | 40.89 (8.77) | 42.74 (6.54) |

¹ Values shown are means with 1 SD in parentheses.

### Electrical Stimulation

The electrical stimulation was provided in accordance with the following parameters: dimension of skin pads for the pair of electrodes: 4″×6″ (10.16 cm×15.24 cm); current intensity: 6 mA; frequency (constant) 1 Hz; symmetrical biphasic current: impulse duration of 500 ms (each impulse having two phases of 250 ms); total charge: 0 Coulomb; maximum power: 0.282 W; maximum energy: 0.0705 J; current density 1.82 mW.cm², with skin pads/pair of electrodes placed on the lateral aspect of the thighs. The placebo treatment was made possible by preparing stimulators forwhich the current generator was disconnected internally from the electrode poles. The stimulators were coded by the manufacturer. The coded stimulators were then matched by the manufacturer to codes given to participants creating the two groups simply named group A and group B (GA and GB). The manufacturer was the only one to know the real status of each stimulator and the group composition. Hence, participants as well as all those working on the study (including researchers, research assistants, research coordinators, trainers and promoters) were blinded as to group assignment (stimulated vs. placebo). At the end of the study, following analysis, the group code was broken and GA was revealed to be the stimulated group (STIM) and GB to be the not-stimulated group (NSTIM).

### Experimental Design

All subjects participated in an interval training exercise program consisting of 30 training sessions over a 10-week period. A typical session lasted 60 minutes: 5 min warm-up, 45 min of interval training, and 10 min of cool down. The training program was comprised of circuits of 6 exercises executed at 65% of maximal capacity interspersed with active rest periods where the level of activity was maintained between 35% and 45% of maximal capacity. The level of activity was monitored through Polar™ heart rate monitors (Polar Electro Canada Inc. TEAM2 PRO SET™) linked in real-time to a computer. The interval training program was designed to be progressive, increasing the level of activity at week 5 of the program by changing the complexity of the exercises and adding free weights as the level of fitness of the participants increased.

### Measures

Prior to and following the study period, the following measurements were made: body weight, and a whole-body DXA image. As stated above, the DXA image provided regional measurements of body fat content, BMC, bone area (BA) and BMD (the ratio of BMC to BA).

### Analysis

The DXA image was divided into 7 regions: head, arms (sum of left and right arms), ribs (sum of left and right ribs), spine, pelvis, legs (sum of left and right legs) and total image. BMC and BA values were taken from the DXA analysis report for each of the regions. The BMC and area values were analyzed using a mixed design ANOVA with time (initial and final values) as the within-subject factor and experimental group (STIM vs. NSTIM) as the between-subjects factor. Results were inspected to identify significant time x group interactions. When such interactions were found, within-group t-tests for correlated means were used to evaluate initial vs. final differences.

### Results

Results are provided below forthe legs, i.e. the region associated with the area of electrical stimulation. Significant differences were found in response between the two groups (a significant group by time interaction) for the bone mineral content (BMC) (Table 7) and bone area (BA) (Table 8). Significant findings are highlighted in bold and underlined.

TABLE 7
Bone Mineral Content in the Legs¹

| Group: | Stim (N = 14) | Non-Stim (N = 15) |
|---|---|---|
| Sample: | | |
| Pre | 1038.64 (223.61) | 1045.40 (242.89) |
| Mid | 1045.79 (220.44) | 1022.67 (221.63) |
| Post | 1056.36 (227.59) | 1032.47 (225.36) |
| Net Change² | 17.71 | −12.93 |
| Net % Change² | 1.71% | −1.24% |
| ANOVA | F | P |
| Time³ | 1.74 | 0.1.85 |
| Group⁴ | 0.03 | 0.875 |
| Group × Time³ | 4.68 | 0.013 |

¹Values shown are in grams. Means are shown with 1 SD in parentheses.
²Change scores calculated as Post value − Pre-value.
³df = 2.54
⁴df = 1.27

TABLE 8
Bone Area in the Legs¹

| Group: | Stim (N = 14) | Non-Stim (N = 14) |
|---|---|---|
| Sample: | | |
| Pre | 813.79 (152.92) | 831.64 (166.28) |
| Mid | 820.50 (161.25) | 804.50 (145.01) |
| Post | 830.14 (170.91) | 817.14 (158.34) |
| Net Change² | 16.36 | −14.50 |
| Net % Change² | 2.01% | −1.74% |
| ANOVA | F | P |
| Time³ | 1.99 | 0.147 |
| Group⁴ | 0.00 | 0.951 |
| Group × Time³ | 4.56 | 0.015 |

¹Values shown are in cm². Means are shown with 1 SD in parentheses.
²Change scores calculated as Post value − Pre-value.
³df = 2.52
⁴df = 1.26

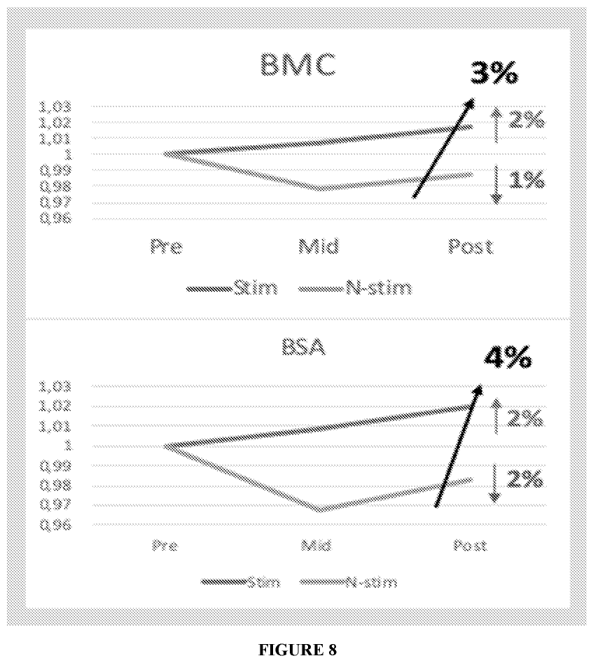

As illustrated in FIG. 8, there was an initial decrease with a subsequent increase in BMC and BA in the Not Stimulated group. This pattern likely reflects bone remodeling pattern seen with an increase in the intensity of exercise where the old bone is removed prior to laying down new bone. At the end of the study period, the bone mass had not yet returned to levels seen prior to the study.

On the other hand, the stimulated group showed continuous increase in both BMC and BA throughout the training program. This suggests the electrical stimulation is associated with continuous bone formation during the training period thereby avoiding the initial period of bone mass decrease associated with exercise-induced remodeling and reducing the risk of any fracture.

During the course of the experiment, BMD is largely unchanged. This points up the value of measuring the components of BMD (BMC & BA) rather than their ratio. Based on the BMD values, one would conclude that there was no net effect of the exercise or stimulation on the bone. By considering the BMC and BA directly, we can see that without stimulation, there is a loss of bone mass and volume, a result that is avoided by electrical stimulation.

### Discussions

In this study, the electrodes were placed to lay over the upper region of the legs (i.e. near the hip). Interestingly, it is this area that showed an increase in BMC and BA in the STIM group while these values decreased in the NSTIM. No other DXA region showed this result (data not shown). This contrast strongly suggests that the beneficial effects are a direct result of the electrical stimulation.

To the best knowledge of the inventors, the present study is the first study to ever demonstrate the effect of electrical stimulation on bone growth in a relatively healthy population.

The present results confirm the uniqueness and efficacy of the electric field that was used in this study, in accordance with the methods and device of the present invention.

### Example 3: In Vitro Studies Using Mice Cell Lines

It is within the skills of those in the art to determine the most effective intensity(ies) and/or the most effective and frequency(ies) of electrical stimulation for bone formation. If desired, in vitro studies may be carried out on one or more of the three types of bone cells: osteoblasts, osteocytes and osteoclasts, as well as on vascular endothelial cells to better define these intensity(ies) and/or frequency(ies), and/or to better understand how, in accordance with the present invention, electrical stimulation can lead to increased bone formation.

For instance, differentiation studies may be carried out using mouse preostoblasts MC3T3-E1 (ATCC, Gaithersburg, MD). Osteocytes proliferation may be studied using mouse osteocytes MLO-Y4 (Kerafast, Boston, MA). These cells may also be used to osteocyte interconnections and peri-osteocytic mineralization.

Effect of low-intensity electric current on osteoclast differentiation and bone resorption may also be evaluated by measuring vitro differentiation of RAW 264.7 (ATCC) macrophage-like cell lines from mouse pre-osteoclasts. Electrical stimulation may also be applied on mouse vascular endothelial cell lines such as 2H-11 (ATCC® CRL-2163™).

As appropriate, these evaluations or measurements may comprise real-time quantitative FOR, analysis of protein expression by Western Blot in cell lysates or by ELISA in supernatants, statistical analysis, etc.

### Example 4: In Vivo Studies in Rats With or Without Metabolic Bone Disease

Animal models may also be used to identify the most effective intensity(ies) and/or the most effective and frequency(ies) of electrical stimulation for bone formation. Animal models may also be used to determine the mechanisms by which electrical stimulation leads to a gain in bone mineral density in vivo. Animals models such as 20-month-old female Wistar rats may also be useful to achieve one or more of the following objectives: 1) assess the effect of electrical stimulation on bone mineral density in aged rats; 2) assess the effect of electrical stimulation: a) on bone microarchitecture and histomorphometric parameters; b) on type H vessels in the bone marrow; c) the expression of bone growth and inhibition factors in bone and bone marrow.

For instance, in one particular protocol the effect of a current of electrical intensity at 6 mA (1 Hz, based on previous in vitro and human studies) for I month may be studied using 20-month-old female Wistar rats. Under such protocol electrical stimulation is done every day at the same time for a duration of 1 h/d to 5/7 days. A group of female rats serves as a control group with respect mainly to biochemical analyses, since the contralateral hip bone of the same rat (which will not have had electrical stimulation(ES)), can be used for comparison of tissue parameters. There is therefore a total of 2 different groups studied: 1. ES×I month 2. Control×I month.

The rats are sacrificed after 1 month of electrical stimulation. Before sacrifice, tetracycline i.p is injected at 2 different periods in order to be able to measure the level of bone formation with bone histomorpho metric. analysis. The femur and the tibia is be removed bilaterally for subsequent analyses. Such analysis may include one or more of: (i) assessment of mineral density and bone microarchitecture (e.g. imaging of ex vivo femurs and tibiae using micro-CT (explore Locus™, GE Healthcare) to measure volumetric mineral density and various microarchitectural parameters of the cortical compartment such as thickness and volume; (ii) evaluation of osteoblastic and osteoclastic activity by bone histomorphometry; (iii) evaluation of type H vessels in the bone marrow using antibodies specific to type H vessels (CD31 and endomucin) in immunohistochemistry and immunofluorescence; (iv) blood analyzes of mineral metabolism and bone markers such as creatinine, calcium, phosphorus, alkaline phosphatase, PTH, osteocalcin, OPG, RANKL, FGF23, sclerostin, P1NP, C-telopeptide, CRP, TNF, L6; (v) qPCR expression and Western Blot protein expression analyzes on bone specimens of NF-K13, NFATc1, osteocalcin, RANKL, OPG Dkk1, SOST, LRP5, β-catenin, Axin2, GSK3, ERK, Akt, HIFI, HIF2a, VEGFA, Notch, SLIT3 and PDGF-BB, and/or TGF-β; (vi) sampling of skin and fat in the stimulated and contralateral region for histological analyzes such as potential macro and microscopic changes in the skin and adipocytes.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “an electrode” includes one or more of such electrodes and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1-59. (canceled)

60. A method for preventing bone loss. promoting bone growth, or healing a bone in a subject, the method comprising:
directly electrically stimulating bone cells with a low-intensity electrical current avoiding muscle contraction;
wherein the low-intensity electrical current is a biphasic current;
administering the electrical stimulation with said low-intensity electrical current in a manner which prevents bone loss and/or heals bones;
wherein said bone cells are selected from a group consisting of arms, legs, pelvis, ribs, trunk, head, spine, wrist, ankle, and whole body;
administering the electrical stimulation with said low-intensity electrical current in a manner which provides at least one of the following benefits: maintenance of bone quality or bone health; prevention of osteoporosis: treatment of osteoporosis; promotion of bone healing; prevention of fractures, treatment of fractures; treatment of spinal fusion; and treatment of spinal-cord injury.

61. The method of claim 60, wherein the low-intensity electrical current has an intensity of about 6.5 mA to about 10.5 mA.

62. The method of claim 60, wherein the low-intensity electrical current comprises a high frequency (HF) modulated by a low frequency (LF).

63. The method of claim 60, wherein the high frequency is about 800 Hz to about 1000 Hz, and the low frequency is about 1 Hz.

64. The method of claim 60. wherein the low-intensity current is selected from the following currents:
1 Hz LF, 6.5 mA, 20/80 duty cycle;
1 Hz LF, 10.5 mA, 20/80 duty cycle;
1 Hz LF, 6.5 mA, 40/60 duty cycle;
1 Hz LF, 10.5 mA, 40/60 duty cycle;
800 Hz HF modulated by 1 Hz LF, 6.5 mA, 20/80 duty cycle;
800 Hz HF modulated by 1 Hz LF, 10.5 mA, 20/80 duty cycle;
1000 Hz HF modulated by 1 Hz LF, 6.5 mA, 20/80 duty cycle; and
1000 Hz HF modulated by 1 Hz LF, 10.5 mA, 20/80 duty cycle.

65. The method of claim 60, wherein the low-intensity current is in accordance with the following parameters: 1 Hz (400 ms ON (200 ms positive (up), 200 ms negative (down), and 600 ms OFF with neutral voltage).

66. The method of claim 60. comprising applying a current of about 6.5 mA to about 10 mA and voltage of about 5 V to about 40 V 5 V to about 40 V on the skin,

67. The method of claim 60, comprising applying a current on the hips of about 5 V to about 40 V.

68. The method of claim 60, comprising providing an electric potential change on a bone cells membrane of about 7 mV to about 70 mV.

69. The method of claim 60, comprising administering the electrical stimulation with said low-intensity electrical current in a manner which provides at least one of the following benefits: promotion of bone growth treatment of fractures; promotion of bone mineralization; promotion bone density; increasing bone surface area; stimulation of osteogenesis via proliferation of H-type vessel endothelial cells; modulation and/or interference in signaling pathways that regulate H-type vessels; promoting release of cytokines and growth factors secreted by vascular endothelial cells; or affecting osteocyte interconnections and peri-osteocytic mineralization.

70. The method of claim 60, comprising:
contacting at least one pair of electrodes on a skin surface over said bone; and
providing to said at least one pair of electrodes a low-intensity electrical current avoiding muscle contraction;
administering said electrical current in a manner which causes stimulation of bone cells.

71. The method of claim 70, comprising administering said low-intensity electrical current in a manner which stimulates proliferation of osteoblasts and/or stimulates differentiation of osteoblasts.

72. The method of claim 70. comprising administering said low-intensity electrical current in a manner which inhibits proliferation of osteoclasts, inhibits differentiation of osteoclasts, inhibits bone resorption controlled by osteoclasts and/or cause an increase in osteocalcin.

73. The method of claim 70. wherein said method is used concurrently with at least one of training exercise(s), at least one bisphosphonate drug, and an osteoporosis medication; and
wherein said at least one bisphosphonate drug is selected from the group consisting of alendronate, ibandronate, risedronate and zoledronic acid, and wherein the osteoporosis medication is selected from the group consisting romosozumab, romosozumab-aggg, raloxifene, bazedoxifene, teriparatide, abaloparatide, denosumab, romosozumab, and menopausal hormone therapy (MHT).

74. The method of claim 60, wherein said electrical stimulation is other than electromagnetic stimulation, other than ultrasound, other than electrolysis, other than sacral neuromodulation (SNM), and other than transcutaneous electrical nerve stimulation (TENS)

75. A device for electrical stimulation of bone tissues, comprising:
an electrical stimulator configured for providing to said bone tissues a low-intensity electrical current avoiding muscle contraction, the electrical stimulator being configured for electrically stimulating directly bone cells via at least one pair of electrodes;
at least one connector adapted to be operatively connected to the at least one pair of electrodes;
at least one of an ON/OFF button, an internal battery, a port, a printed circuit board;
at least one pair of electrodes to be contacted with a skin surface over said bone;
at least one pair of electrodes comprises an anode and a cathode made of carbon;
wherein said at least one pair of electrodes are incorporated into a skin pad; and
wherein said at least one pair of electrodes comprises an electrical wire and a connector for connecting with the device and transporting the electrical current therefrom.

76. The device according to claim 75, wherein said at least one pair of electrodes is introduced within a garment and/or incorporated to a conductive fabric; and
wherein said garment is selected from the group consisting of a shirt, a sleeveless shirt, a vest, a long pant, a short pant, a sock, a glove, an elbow sleeve, a back belt, a brace, and a wrist bracelet.

77. The device according to claim 75, wherein said electrical stimulation is other than electromagnetic stimulation, other than ultrasound, other than electrolysis, other than sacral neuromodulation (SNM), and other than transcutaneous electrical nerve stimulation (TENS).

78. The device of claim 75 being configured or designed to output the low-intensity current according to at least one of:
1 Hz LF, 6.5 mA, 20/80 duty cycle;
1 Hz LF, 10.5 mA, 20/80 duty cycle;
1 Hz LF, 6.5 mA, 40/60 duty cycle;
1 Hz LF, 10.5 mA, 40/60 duty cycle;
800 Hz HF modulated by 1 Hz LF, 6.5 mA, 20/80 duty cycle;
800 Hz HF modulated by 1 Hz LF, 10.5 mA, 20/80 duty cycle;
1000 Hz HF modulated by 1 Hz LF, 6.5 mA, 20/80 duty cycle; and
1000 Hz HF modulated by 1 Hz LF, 10.5 mA, 20/80 duty cycle.

79. The device according to claim 75 being configured or designed to:
output said low-intensity electrical current in a manner which stimulates proliferation of osteoblasts and/or stimulates differentiation of osteoblasts; and
output said low-intensity electrical current in a manner which inhibits proliferation of osteoclasts, inhibits differentiation of osteoclasts, inhibits bone resorption controlled by osteoclasts and/or cause an increase in osteocalcin.
